(19) United States
(12) Patent Application Publication
(10) Pub. No.: US 2003/0092737 A1
(43) Pub. Date: May 15, 2003

(54) Combination of active ingredients for the treatment of senile dementia of the Alzheimer type

(76) Inventors: Maffrand Jean Pierre, Portet sur Garonne (FR); Philippe Soubrie, Saint Matthieu de Treviers (FR); Jean Paul Terranova, Saint Gely du Fesc (FR)

Correspondence Address: SANOFI-SYNTHELABO INC., 9 GREAT VALLEY PARKWAY, P.O. BOX 3026, MALVERN, PA, 19355, US

(21) Appl. No.: 10/268,378

(22) Filed: Oct. 10, 2002

Related U.S. Application Data
(63) Continuation of application No. 09/554,139, filed on Jun. 29, 2000, now abandoned.
Application No. 09/554,139 is a 371 of international application No. PCT/FR98/02384, filed on Nov. 9, 1998.

(30) Foreign Application Priority Data
Nov. 14, 1997 (FR) 9714322
Nov. 14, 1997 (FR) 9714324

(51) Int. Cl. A61K 31/444; A61K 31/44

(52) U.S. Cl. 514/334; 514/357

(57) ABSTRACT

A pharmaceutical composition containing a compound (a) selected from 1-(2-naphth-2-ylethyl)-4-(3-trifluoro-methylphenyl)-1,2,3,6-tetrahydropryidine and a 4-substituted 1-phenylalkyl-1,2,3,6-tetrahydropyridine in combination with a compound (b) active in the symptomatic treatment of dementia of the Alzheimer type (DAT), especially an acetyicholinesterase inhibitor, for the complete treatment of DAT.

The object of the present invention is a pharmaceutical composition containing a novel combination of active ingredients for the treatment of senile dementia of the Alzheimer type, constituted of 1,2,3,6-tetrahydropyridine derivatives, optionally in the form of one of their pharmaceutically acceptable salts and a substance active in the symptomatic treatment of senile dementia of the Alzheimer type, in particular an acetylcholinesterase inhibitor, optionally in the form of one of its pharmaceutically acceptable salts and its use for the preparation of medicines designed for the treatment of senile dementia of the Alzheimer type.

Senile dementia of the Alzheimer type designated hereafter DAT (“dementia of the Alzheimer type”) is a neurodegenerative disease characterized clinically by the progressive degeneration of cognitive functions, occurring in elderly people with an incidence which increases with age. In the light of demographic trends DAT will become an increasingly widespread disease.

A reduction of the level of several neurotransmitters, of acetylcholine in particular, has been observed in patients suffering from DAT

The only treatment for DAT currently available commercially consists of administering acetylcholinesterase inhibitors which by reducing the hydrolysis of acetylcholine thus increase its bioavailability. Hence it is a symptomatic treatment.

Tacrine, marketed under the trade mark COGNEX®, and donepezil, sold under the trade mark ARICEPT®, are acetylcholinesterase inhibitors indicated for the symptomatic treatment of mild to moderate forms of DAT. Other products for the symptomatic treatment of DAT are under study. Some of them also act on the availability of acetylcholine, others improve the symptomatology of patients suffering from DAT by other mechanisms. Hitherto, no commercially available medicine has proved capable of slowing the progression of the disease. EP-458696 describes the use of 1-(2-naphth-2-ylethyl)-4-(3-trifluoromethylphenyl)-1,2,3,6-tetrahydropyridine, designated in the literature SR 57746, for the preparation of medicines designed to combat neurodegenerative states, including senile dementia and Alzheimer's disease. The neurotrophic action of SR 57746 on the nervous system is similar to that of certain endogenous neurotrophins such as, for example, the nerve growth factor (NGF).

WO 97/01536 describes novel 4-substituted 1-phenylalkyl-1,2,3,6-tetrahydropyridines having a neuroprotective and neurotrophic activity similar to that of certain endogenous neurotrophins. As a result of this activity, it is presumed that the compounds described in this patent application will be useful in the treatment of several diseases of the central nervous system, including Alzheimer's disease.

The activity of the compound SR 57746 and the compounds described in WO 97/01536 in the treatment of the nervous diseases such as DAT is not designed to treat the symptoms but, by protecting the neurones, to modify the course of the disease and to reduce its progression.

It has now been found that the combination of the above compounds, optionally in the form of one of their pharmaceutically acceptable salts, with a compound active in the symptomatic treatment of senile dementia of the Alzheimer type, in particular an acetylcholinesterase inhibitor, optionally in the form of one of its pharmaceutically acceptable salts, leads to a complete and very efficacious treatment of DAT, the combination exerting a rapid and complementary effect.

Thus, the object of the present invention is a pharmaceutical composition containing as active ingredients

a compound (a) selected from 1-(2-naphth-2-ylethyl)-4-(3-trifluoromethylphenyl)-1,2,3,6-tetrahydropyridine and a compound of formula (I):

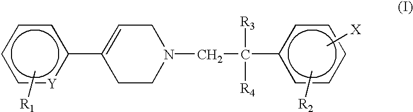

in which

Y is —CH— or —N—;

R₁ hydrogen, halogen, a CF₃, (C₃-C₄) alkyl or (C₁-C₄) alkoxy group;

R₂ is hydrogen, halogen, hydroxyl, CF₃, (C₃-C₄) alkyl or (C₁-C₄) alkoxy group;

R₃ and R₄ each is hydrogen or (C₁-C₃) alkyl;

X is

(a) (C₃-C₆) alkyl; (C₃-C₆) alkoxy; (C₃-C₇) carboxyalkyl; (C₁-C₄) alkoxycarbonyl (C₃-C₆) alkyl; (C₃-C₇) carboxyalkoxy; or (C₁-C₄) alkoxycarbonyl (C₃-C₆) alkoxy;

(b) a radical selected from (C₃-C₇) cycloalkyl, (C₃-C₇) cycloalkyloxy, (C₃-C₇) cycloalkylmethyl, (C₃-C₇) cycloalkylamino and cyclohexenyl, said radical being optionally substituted by halogen, hydroxy, (C₁-C₄) alkoxy, carboxy, (C₁-C₄) alkoxycarbonyl, amino, mono or di-(C₁-C₄) alkylamino; or

(c) a group selected from phenyl, phenoxy, phenylamino, N-(C₁-C₃) alkylphenylamino, phenylmethyl, phenylethyl, phenylcarbonyl, phenylthio, phenylsulfonyl, phenylsulfinyl or styryl, said phenyl group being optionally mono- or polysubstituted by halogen, CF₃, (C₁-C₄) alkyl, (C₁-C₄) alkoxy, cyano, amino, mono- or di-(C₁-C₄) alkylamino, (C₁-C₄) acylamino, carboxy, (C₁-C₄) alkoxycarbonyl, aminocarbonyl, mono- or di-(C₁-C₄) alkylaminocarbonyl, amino (C₁-C₄) alkyl, hydroxy (C₁-C₄) alkyl or halogeno (C₁-C₄) alkyl;

optionally in the form of one of its pharmaceutically acceptable salts and

a compound (b) active in the symptomatic treatment of DAT, optionally in the form of one of its pharmaceutically acceptable salts provided that when compound (a) is other than 1-(2-naphth-2-ylethyl)-4-(3-trifluoromethylphenyl)-1,2,3,6-tetrahydropyridine or one of its pharmaceutically acceptable salts, compound (b) is an acetylcholinesterase inhibitor.

1-(2-naphth-2-ylethyl)-4-(3-trifluoromethylphenyl)-1,2,3,6-tetrahydropyridine (SR 57746) was described in EP 101 381 and the compounds of formula (I) above are described in WO 97/01536.

A particularly advantageous compound (a) is 1-(2-naphth-2-ylethyl)-4-(3-trifluoromethylphenyl)-1,2,3,6-tetrahydropyridine (SR 57746), optionally in the form of one of itsq pharmaceutically acceptable salts.

Of the pharmaceutically acceptable salts of SR 57746, the hydrochloride designated hereafter SR 57746A is a particularly preferred salt.

An advantageous method for the preparation of SR 57746A consists of the reaction between 2-(2-bromoethyl) naphthalene and 4-(3-trifluoromethylphenyl) -1,2,3,6-tetrahydropyridine and the isolation of 1-(2-naphth-2-ylethyl)-4-(3-trifluoromethylphenyl)-1,2,3,6-tetrahydropyridine hydrochloride which is then crystallized from an ethanol/water mixture by heating and cooling to 5° C. with a rate of cooling of 10° C./hour and a stirring speed of 400 revolutions/minute, so as to obtain a mixture of two crystalline forms in a ratio of about 66/34.

SR 57746A is preferably used in a microparticulate form, for example in an essentially amorphous form obtained by spray drying or in a microcrystalline form by micronization.

Another particularly advantageous compound (a) is 1-{2-(4-biphenylyl)ethyl}-4-(3-trifluoromethylphenyl)-1,2,3,6-tetrahydro-pyridine, in particular its hydrochloride salt.

Other advantageous compounds are the following:

1-{2-(3′-chloro-4-biphenylyl)ethyl}-4-(3-trifluoromethylphenyl)-1,2,3,6-tetrahydropyridine;

1-{2-(2′-chloro-4-biphenylyl)ethyl}-4-(3-trifluoromethylphenyl)-1,2,3,6-tetrahydropyridine;

1-{2-(4′-chloro-4-biphenylyl)ethyl}-4-(3-trifluoromethylphenyl)-1,2,3,6-tetrahydropyridine;

1-{2-(4′-fluoro-4-biphenylyl)ethyl}-4- (3-trifluoromethylphenyl)-1,2,3,6-tetrahydropyridine;

1-{2-(3′-trifluoromethyl-4-biphenylyl)ethyl}-4-(3-trifluoromethyl-phenyl)-1,2,3,6-tetrahydropyridine;

1-{2- (4-cyclohexylphenyl)ethyl}-4- (3-trifluoromethylphenyl)-1,2,3,6-tetrahydropyridine;

1-{2-(4-biphenylyl)ethyl}-4-(4-fluorophenyl)-1,2,3,6-tetrahydro-pyridine;

1-{2-(4-biphenylyl)-2-methylpropyl}-4-(3-trifluoromethylphenyl)-1,2,3,6-tetrahydropyridine;

1-{2-(4-phenoxyphenyl)ethyl}-4-(3-trifluoromethylphenyl)-1,2,3,6-tetrahydropyridine;

1-{2- (4-benzylphenyl)ethyl}-4-(3-trifluoromethylphenyl)-1,2,3,6-tetrahydropyridine;

1-{2-(4-n-butylphenyl)ethyl}-4-(3-trifluoromethylphenyl)-1,2,3,6-tetrahydropyridine;

1-{2- (4-n-butoxyphenyl)ethyl}-4-(3-trifluoromethylphenyl)-1,2,3,6-tetrahydropyridine;

1-{2- (4-(4-ethoxycarbonylpropoxy)phenyl)ethyl}-4-(3-trifluoro-methylphenyl)-1,2,3,6-tetrahydropyridine;

1-{2-(4-biphenylyl)ethyl}-4-(6-chloropyrid-2-yl)-1,2,3,6-tetrahydro-pyridine;

1-{2-(2,3′-dichloro-4-biphenylyl)ethyl}-4-(3-trifluoromethylphenyl)-1,2,3,6-tetrahydropyridine;

1-{2-(3-chloro-4-biphenylyl)ethyl}-4-(3-trifluoromethylphenyl)-1,2,3,6-tetrahydropyridine;

1-{2-(3′,5′-dichloro-4-biphenylyl)ethyl}-4-(3-trifluoromethyl-phenyl)-1,2,3,6-tetrahydropyridine;

1-{2-(2′,4′-dichloro-4-biphenylyl)ethyl}-4-(3-trifluoromethyl-phenyl)-1,2,3,6-tetrahydropyridine;

1-{2-(2-chloro-4-biphenylyl)ethyl}-4-(3-trifluoromethylphenyl)-1,2,3,6-tetrahydropyridine;

1-{2-(3′-chloro-4-biphenylyl)-2-methylpropyl}-4-(3-trifluoromethylphenyl)-1,2,3,6-tetrahydropyridine;

1-{2-(2-fluoro-4-biphenylyl)propyl}-4-(3-trifluoromethylphenyl)-1,2,3,6-tetrahydropyridine;

1-{2-(4-methoxy-3-biphenylyl)ethyl}-4-(3-trifluoromethylphenyl)-1,2,3,6-tetrahydropyridine;

1-{2-(4¹-methoxy-4-biphenylyl)ethyl}-4-(3-trifluoromethylphenyl)-1,2,3,6-tetrahydropyridine;

1-{2-(4′-hydroxy-4-biphenylyl)ethyl}-4-(3-trifluoromethylphenyl)-1,2,3,6-tetrahydropyridine;

1-{2-(4′-ethoxycarbonylbutoxy-4-biphenylyl)ethyl}-4-(3-trifluoro-methylphenyl)-1,2,3,6-tetrahydropyridine;

1-{2-(3-biphenylyl)ethyl}-4-(3-trifluoromethylphenyl)-1,2,3,6-tetrahydropyridine;

1-{2-(3′-chloro-4′-fluoro-4-biphenylyl)ethyl}-4-(3-trifluoromethyl-phenyl)-1,2,3,6-tetrahydropyridine;

1-{2-(2′-trifluoromethyl-4-biphenylyl)ethyl}-4-(3-trifluoromethyl-phenyl)-1,2,3,6-tetrahydropyridine;

1-{2-(3,4-diisobutylphenyl)ethyl}-4-(3-trifluoromethylphenyl)-1,2,3,6-tetrahydropyridine;

1-{2-(3,4-dipropylphenyl)ethyl}-4-(3-trifluoromethylphenyl)-1,2,3,6-tetrahydropyridine;

1-{2-(4-cyclohexylphenyl)ethyl}-4-(6-chloropyrid-2-yl)-1,2,3,6-tetrahydropyridine;

1-{2-(4-isobutylphenyl)propyl}-4-(6-chloropyrid-2-yl)-1,2,3,6-tetrahydropyridine;

and their pharmaceutically acceptable salts.

In the present description, the expression “compound active in the symptomatic treatment of DAT” designates a product which is capable of improving the symptomatology of patients suffering from DAT without having an effect on the causes of the disease.

Such compounds are, for example, acetylcholinesterase inhibitors, M₁ muscarinic agonists, nicotinic agonists N-methyl-D-aspartate (NMDA) receptor antagonists, nootropics, the acetylcholinesterase inhibitors being particularly advantageous.

In accordance with a preferred feature, the invention relates to a pharmaceutical composition containing as active ingredient a compound (a), optionally in the form of one of its pharmaceutically acceptable salts and a compound (b) selected from the acetylcholinesterase inhibitors, optionally in the form of one of its pharmaceutically acceptable salts.

Particularly advantageous acetylcholinesterase inhibitors are tacrine and donepezil.

Other acetylcholinesterase inhibitors which may be used are for example rivastigmine (SDZ-ENA-713), galanthamine, metrifonate, eptastigmine, velnacrine, physostigmine (Drugs, 1997, 53 (5): 752-768; The Merck Index 12 ed.).

Other acetylcholinesterase inhibitors are 5,7-dihydro-3-{2-(1-(phenylmethyl)-4-piperidinyl)ethyl}-6H-pyrrolo (3,2-f)-1,2-benzisoxazol-6-one, also known as icopezil (J. Med. Chem., 1995, 38: 2802-2808), MDL-73,745 or zifrosilone (Eur. J. Pharmacol., 1995, 276: 93-99), TAK-147 (J. Med. Chem., 1994, 37: 2292-2299).

Other acetylcholinesterase inhibitors are, for example those which are described in the patent applications JP 09-095483, WO 97/13754, WO 97/21681, WO 97/19929, ZA 96-04565, U.S. Pat. No. 5,455,245, WO 95-21822, EP 637 586, U.S. Pat. No. 5,401,749, EP 742 207, U.S. Pat. No. 5,547,960, WO 96/20176, WO 96/02524, EP 677 516, JP 07-188177, JP 07-133274, EP 649 846, EP 648 771, JP 07-048370, U.S. Pat. No. 5,391,553, WO 94/29272, EP 627 400.

M₁ receptor antagonists are, for example, milameline, besipiridine, talsaclidine, xanomeline, YM-796 and YM-954 (Eur. J. Pharmacol., 1990, 187: 479-486), 3-{N-(2-diethylamino-2-methylpropyl)-6-phenyl-5-propyl}-pyridazinamine, also known as SR-46559 (Biorg. Med. Chem. Let., 1992, 2: 833-838), AF-102, CI-979, L-689,660, LU 25-109, S-99 77-2, SB 202,026, thiopilocarpine, WAL 2014 (Pharmacol. Toxicol., 1996, 78: 59-68).

Advantageous nicotine agonists are for example MKC-231 (Biorg. Med. Chem. Let., 1995, 5 (14): 1495-1500), T-588 (Japan J. Pharmacol., 1993, 62: 81-86), ABT-418 (Br. J. Pharmacol., 1997, 120: 429-438).

An advantageous NMDA receptor antagonist is for example memantine (Arzneim. Forsch., 1991, 41: 773-780).

In accordance with another feature, the present invention relates to the use of the compositions of the invention for the preparation of medicines designed for the treatment of senile dementia of the Alzheimer type.

In accordance with another feature the present invention also relates to another method for the treatment of senile dementia of the Alzheimer type which consists of administering to a patient suffering from this disease an efficacious dose of a compound (a) above, optionally in the form of one of its pharmaceutically acceptable salts and an efficacious dose of a compound (b), in particular an acetylcholinesterase inhibitor, optionally in the form of one of its pharmaceutically acceptable salts, said administrations being simultaneous, sequential or alternating at intervals and the efficacious doses of the active ingredients being contained in separate unit forms of administration or, when the active ingredients are administered simultaneously, the two active ingredients being advantageously contained in a single pharmaceutical form.

The active ingredients according to the present invention are preferably administered orally.

In the pharmaceutical compositions of the present invention for oral administration, the active ingredients may be administered in unit forms of administration, in a mixture with standard pharmaceutical vehicles, to animals and to human beings for the treatment of the above-mentioned diseases. The appropriate unit forms of administration include for example optionally divisible tablets, capsules, powders, granules and solutions or oral suspensions.

When a solid composition is prepared in the form of tablets, the principal active ingredient is mixed with a pharmaceutical vehicle such as gelatin, starch, lactose, magnesium stearate, talc, gum arabic or the like. The tablets may be coated with sucrose or other suitable materials or they may also be treated so that they have a prolonged or delayed activity and that they continuously release a predefined quantity of active ingredient.

A preparation of capsules is obtained by mixing the active ingredient with a diluent and by pouring the mixture obtained into soft or hard capsules.

A preparation in the form of a syrup or elixir may contain the active ingredient together with a sweetening agent, preferably calorie-free, methylparaben and propylparaben as antiseptics as well as a flavouring agent and a suitable colouring matter.

The powders and granules dispersible in water may contain the active ingredient in a mixture with dispersion agents or wetting agents, or suspension agents like polyvinylpyrrolidone, just as with sweetening agents or flavour correctors.

The active ingredient may also be formulated in the form of microcapsules, optionally with one or more vehicles or additives.

In the pharmaceutical compositions according to the present invention, the active ingredient may also be in the form of an inclusion complex in cyclodextrins, their ethers or their esters.

The quantity of active ingredient to be administered depends, as always, on the degree of advancement of the disease as well as on the age and weight of the patient.

The doses of the two active ingredients are similar to those usually selected in the state of the art for the isolated administration of each of these active ingredients.

The compositions according to the invention thus contain recommended doses for the uncombined treatments, for example, of 0.5 mg to 700 mg of compound (a) or of one of its pharmaceutically acceptable salts and 0.1 to 50 mg of compound (b) or of one of its pharmaceutically acceptable salts or even lower doses, given that the combination exerts a synergistic effect.

Advantageous compositions contain for example 0.5 to 5 mg of SR 57746 or one of its pharmaceutically acceptable salts and 0.1 to 50 mg of an acetylcholinesterase inhibitor or one of its pharmaceutically acceptable salts.

Preferred compositions contain 0.5 to 5 mg of SR 57746 or one of its, pharmaceutically acceptable salts, in particular the hydrochloride, and 2 to 10 mg of donepezil or one of its pharmaceutically acceptable salts.

The doses indicated in the present prescription refer to the active ingredients not combined in salt form.

The activity of the composition according to the invention was demonstrated by using a specific model for the septo-hippocampal cholinergic system on lesions caused by the injection of vincristine which induces biochemical alterations similar to the changes present in Alzheimer's disease.

The procedures used in this model, lesions caused by vincristine as well as the evaluation of the social memory are described in EP 655247.

Evaluation Test of the Social Memory in the Rat.

After lesions have provoked by injection of vincristine as described in EP 655247 the rats exhibit a stable and durable amnesia. The rats are divided into two groups, one group receiving solvent and the other group receiving SR 57746A at a dose of 5 mg.kg p.o., a dose which is insufficient to permit functional recovery in terms of memory in the rats undergoing this test (the efficacious dose being 10 mg/kg as described in EP 655247). The dose of 1 mg/kg i.p. of tacrine is then administered to the two groups of rats. The control group which has received solvent and tacrine shows no recovery of memory whereas the group which has been treated with SR 57746A (sub-efficacious dose) and tacrine shows a significant recovery of memory retention deficits.

The results of this test indicate a synergistic action of the combination of the present invention.

As a result of this complementary and synergistic effect of the constituents of the combination, simultaneously guaranteeing the protection and even cure of the neurones affected by the disease as well as the immediate improvement of the symptoms in the patient, the composition of the invention makes possible an efficacious treatment of DAT in all its forms.

1. A pharmaceutical composition containing as active ingredients
a compound (a) selected from 1-(2-naphth-2-ylethyl)-4-(3-trifluoromethylphenyl)-1,2,3,6-tetrahydropyridine and a compound of formula (I): 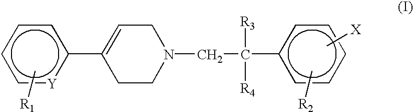
in which
Y is —CH— or —N—;
R₁ is hydrogen, halogen, a CF₃, (C₃-C₄) alkyl or (C₁-C₄) alkoxy group;
R₂ is hydrogen, halogen, hydroxyl, CF₃, (C₃-C₄) alkyl or (C₁-C₄) alkoxy group;
R₃ and R₄ each is hydrogen or (C₁-C₃) alkyl;
X is
(a) (C₃-C₆) alkyl; (C₃-C₆) alkoxy; (C₃-C₇) carboxyalkyl; (C₁-C₄) alkoxycarbonyl (C₃-C₆) alkyl; (C₃-C₇) carboxyalkoxy; or (C₁-C₄) alkoxycarbonyl (C₃-C₆) alkoxy;
(b) a radical selected from (C₃-C₇) cycloalkyl, (C₃-C₇) cycloalkyloxy, (C₃-C₇) cycloalkylmethyl, (C₃-C₇) cycloalkylamino and cyclohexenyl, said radical being optionally substituted by halogen, hydroxy, (C₁-C₄) alkoxy, carboxy, (C₁-C₄) alkoxycarbonyl, amino, mono or di-(C₁-C₄) alkylamino; or
(c) a group selected from phenyl, phenoxy, phenylamino, N-(C₁-C₃) alkylphenylamino, phenylmethyl, phenylethyl, phenylcarbonyl, phenylthio, phenylsulfonyl, phenylsulfinyl or styryl, said phenyl group being optionally mono- or polysubstituted by halogen, CF₃, (C₁-C₄) alkyl, (C₁-C₄) alkoxy, cyano, amino, mono- or di-(C₁-C₄) alkylamino, (C₁-C₄) acylamino, carboxy, (C₁-C₄) alkoxycarbonyl, aminocarbonyl, mono- or di-(C₁-C₄) alkylaminocarbonyl, amino (C₁-C₄) -alkyl, hydroxy (C₁-C₄) alkyl or halogeno (C₁-C₄) alkyl;
optionally in the form of one of its pharmaceutically acceptable, salts and
a compound (b) active in the symptomatic treatment of DAT, optionally in the form of one of its pharmaceutically acceptable salts provided that when compound (a) is other than 1-(2-naphth-2-ylethyl)-4-(3-trifluoromethylphenyl)-1,2,3,6-tetrahydropyridine or one of its pharmaceutically acceptable salts, compound (b) is an acetylcholinesterase inhibitor.

2. Composition according to claim 1, characterized in that it contains as active ingredients 1-(2-naphth-2-ylethyl)-4-(3-trifluoromethylphenyl)-1,2,3,6-tetrahydropyridine, optionally in the form of one of its pharmaceutically acceptable salts in combination with a compound active in the symptomatic treatment of senile dementia of the Alzheimer type, optionally in the form of one of its pharmaceutically acceptable salts.

3. Composition according to claim 1, characterized in that it contains as active ingredients
a compound of formula (I): 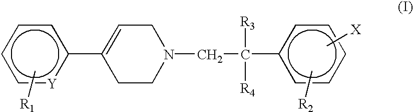
in which
Y is —CH— or —N—;
R₁ is hydrogen, halogen, a CF₃, (C₃-C₄) alkyl or (C₁-C₄) alkoxy group;
R₂ is hydrogen, halogen, hydroxyl, CF₃, (C₃-C₄) alkyl or (C₁-C₄) alkoxy group;
R₃ and R₄ each is hydrogen or (C₁-C₃) alkyl;
X is
(a) (C₃-C₆) alkyl; (C₃-C₆) alkoxy; (C₃-C₇) carboxyalkyl; (C₁-C₄) alkoxycarbonyl (C₃-C₆) alkyl; (C₃-C₇) carboxyalkoxy; or (C₁-C₄) alkoxycarbonyl (C₃-C₆) alkoxy;
(b) a radical selected from (C₃-C₇) cycloalkyl, (C₃-C₇) cycloalkyloxy, (C₃-C₇) cycloalkylmethyl, (C₃-C₇) cycloalkylamino and cyclohexenyl, said radical being optionally substituted by halogen, hydroxy, (C₁-C₄) alkoxy, carboxy, (C₁-C₄) alkoxycarbonyl, amino, mono or di-(C₁-C₄) alkylamino; or
(c) a group selected from phenyl, phenoxy, phenylamino, N-(C₁-C₃) alkylphenylamino, phenylmethyl, phenylethyl, phenylcarbonyl, phenylthio, phenylsulfonyl, phenylsulfinyl or styryl, said phenyl group being optionally mono- or polysubstituted by halogen, CF₃, (C₁-C₄) alkyl; (C₁-C₄) alkoxy, cyano, amino, mono- or di-(C₁-C₄) alkylamino, (C₁-C₄) acylamino, carboxy, (C₁-C₄) alkoxycarbonyl, aminocarbonyl, mono- or di-(C₁-C₄) alkylaminocarbonyl, amino (C₁-C₄) alkyl, hydroxy (C₁-C₄) alkyl or halogeno (C₁-C₄) alkyl;
optionally in the form of one of its pharmaceutically acceptable salts and
an acetylcholinesterase inhibitor,
or a pharmaceutically acceptable salt of the latter.

4. Composition according to claim 3, characterized in that compound (a) is 1-{2-(4-biphenylyl)ethyl}-4-(3-trifluoro-methylphenyl)-1,2,3,6-tetrahydropyridine or its hydrochloride salt.

5. Composition according to claim 3, characterized in that compound (a) is selected from the following compounds:
1-{2-(3′-chloro-4-biphenylyl)ethyl}-4-(3-trifluoromethylphenyl)-1,2,3,6-tetrahydropyridine;
1-{2-(2′-chloro-4-biphenylyl)ethyl}-4-(3-trifluoromethylphenyl)-1,2,3,6-tetrahydropyridine;
1-{2-(4′-chloro-4-biphenylyl)ethyl}-4-(3-trifluoromethylphenyl)-1,2,3,6-tetrahydropyridine;
1-{2-(4′-fluoro-4-biphenylyl)ethyl}-4-(3-trifluoromethylphenyl)-1,2,3,6-tetrahydropyridine;
1-{2-(3′-trifluoromethyl-4-biphenylyl)ethyl}-4-(3-trifluoromethyl-phenyl)-1,2,3,6-tetrahydropyridine;
1-{2-(4-cyclohexylphenyl)ethyl}-4-(3-trifluoromethylphenyl)-1,2,3,6-tetrahydropyridine;
1-{2-(4-biphenylyl)ethyl}-4-(4-fluorophenyl)-1,2,3,6-tetrahydro-pyridine;
1-{2-(4-biphenylyl)-2-methylpropyl}-4-(3-trifluoromethylphenyl)-1,2,3,6-tetrahydropyridine;
1-{2-(4-phenoxyphenyl)ethyl}-4-(3-trifluoromethylphenyl)-1,2,3,6-tetrahydropyridine;
1-{2-(4-benzylphenyl)ethyl}-4-(3-trifluoromethylphenyl)-1,2,3,6-tetrahydropyridine;
1-{2-(4-n-butylphenyl)ethyl}-4-(3-trifluoromethylphenyl)-1,2,3,6-tetrahydropyridine;
1-{2-(4-n-butoxyphenyl)ethyl}-4-(3-trifluoromethylphenyl)-1,2,3,6-tetrahydropyridine;
1-{2-(4-(4-ethoxycarbonylpropoxy)phenyl)ethyl}-4-(3-trifluoro-methylphenyl)-1,2,3,6-tetrahydropyridine;
1-{2-(4-biphenylyl)ethyl}-4-(6-chloropyrid-2-yl)-1,2,3,6-tetrahydro-pyridine;
1-{2-(2,3′-dichloro-4-biphenylyl)ethyl}-4-(3-trifluoromethylphenyl)-1,2,3,6-tetrahydropyridine;
1-{2-(3-chloro-4-biphenylyl)ethyl}-4-(3-trifluoromethylphenyl)-1,2,3,6-tetrahydropyridine;
1-{2-(3′,5-dichloro-4-biphenylyl)ethyl}-4-(3-trifluoromethyl-phenyl)-1,2,3,6-tetrahydropyridine;
1-{2-(2′,4′-dichloro-4-biphenylyl)ethyl}-4- (3-trifluoromethyl-phenyl)-1,2,3,6-tetrahydropyridine;
1-{2-(2-chloro-4-biphenylyl)ethyl}-4-(3-trifluoromethylphenyl)-1,2,3,6-tetrahydropyridine;
1-{2-(3′-chloro-4-biphenylyl) -2-methylpropyl}-4-(3-trifluoromethylphenyl)-1,2,3,6-tetrahydropyridine;
1-{2-(2-fluoro-4-biphenylyl)propyl}-4-(3-trifluoromethylphenyl)-1,2,3,6-tetrahydropyridine;
1-{2-(4-methoxy-3-biphenylyl)ethyl}-4-(3-trifluoromethylphenyl)-1,2,3,6-tetrahydropyridine;
1-{2-(4′-methoxy-4-biphenylyl)ethyl}-4-(3-trifluoromethylphenyl)-1,2,3,6-tetrahydropyridine;
1-{2-(4′-hydroxy-4-biphenylyl)ethyl}-4-(3-trifluoromethylphenyl)-1,2,3,6-tetrahydropyridine;
1-{2-(4′-ethoxycarbonylbutoxy-4-biphenylyl)ethyl}-4- (3-trifluoro-methylphenyl)-1,2,3,6-tetrahydropyridine;
1-{2-(3-biphenylyl)ethyl}-4-(3-trifluoromethylphenyl)-1,2,3,6-tetrahydropyridine;
1-{2-(3′-chloro-4′-fluoro-4-biphenylyl)ethyl}-4-(3-trifluoromethyl-phenyl)-1,2,3,6-tetrahydropyridine;
1-{2-(2′-trifluoromethyl-4-biphenylyl)ethyl}-4-(3-trifluoromethyl-phenyl)-1,2,3,6-tetrahydropyridine;
1-{2-(3,4-diisobutylphenyl)ethyl}-4-(3-trifluoromethylphenyl)-1,2,3,6-tetrahydropyridine;
1-{2-(3,4-dipropylphenyl)ethyl}-4-(3-trifluoromethylphenyl)-1,2,3,6-tetrahydropyridine;
1-{2-(4-cyclohexylphenyl)ethyl}-4-(6-chloropyrid-2-yl)-1,2,3,6-tetrahydropyridine;
1-{2-(4-isobutylphenyl)propyl}-4-(6-chloropyrid-2-yl)-1,2,3,6-tetrahydropyridine;
and their pharmaceutically acceptable salts.

6. Composition according to claim 1 characterized in that the compound active in the symptomatic treatment of senile dementia of the Alzheimer type is selected from acetylcholinesterase inhibitors, M₁ muscarinic agonists, nicotinic agonists, NMDA receptor antagonists and nootropic agents.

7. Composition according to claim 6, characterized in that compound (b) is an acetylcholinesterase inhibitor.

8. Composition according to claim 7, characterized in that the acetylcholinesterase inhibitor is selected from tacrine and donepezil.

9. Composition according to claim 7, characterized in that the acetylcholinesterase inhibitor is selected from rivastigmine, galanthamine, metrifonate, eptastigmine, velnacrine, phystostigmine, icozepil and zifrosilone.

10. Composition according to claim 1, characterized in that it contains from 0.5 to 700 mg of compound (a).

11. Composition according to claim 1, characterized in that it contains from 0.1 to 50 mg of compound (b).

12. Composition according to claim 2, characterized in that it contains from 0.5 to 10 mg of 1-(2-naphth-2-ylethyl)-4-(3-trifluoro-methylphenyl)-1,2,3,6-tetrahydropyridine.

13. Composition according to claim 2, characterized in that it contains as active ingredients 1-(2-naphth-2-ylethyl)-4-(3-trifluoromethylphenyl)-1,2,3,6-tetrahydropyridine and donepezil or their pharmaceutically acceptable salts.

14. Composition according to claim 3, characterized in that it contains as active ingredients 1-{2-(4-biphenylyl)ethyl}4-(3-trifluoromethylphenyl)-1,2,3,6-tetrahydropyridine and donepezil or their pharmaceutically acceptable salts.

15. Composition according to claim 2 containing 0.5 to 5 mg of 1-(2-naphth-2-ylethyl)-4-(3-trifluoromethylphenyl)-1,2,3,6-tetrahydropyridine and 2 to 10 mg of donepezil.

16. Composition according to any one of the preceding claims for the treatment of senile dementia of the Alzheimer type.

17. Use of the composition according to any one of the preceding claims for the preparation of medicines designed for the treatment of senile dementia of the Alzheimer type.

18. Use according to claim 17, characterized in that the compound (a) is selected from 1-(2′-naphth-2-ylethyl)-4-(3-trifluoromethylphenyl)-1,2,3,6-tetrahydropyridine and 1-{2-(4-biphenylyl)ethyl}-4-(3-trifluoromethylphenyl)-1,2,3,6-tetrahydropyridine.

19. Use according to claim 17, characterized in that the compound (b) is selected from tacrine and donepezil.
